(19) United States
(12) Patent Application Publication
(10) Pub. No.: US 2021/0027889 A1
(43) Pub. Date: Jan. 28, 2021

(54) System and Methods for Predicting Identifiers Using Machine-Learned Techniques

(71) Applicant: Hank.AI, Inc., Hendersonville, NC (US)

(72) Inventors: Jack Edward Neil, Hendersonville, NC (US); Sergey Razin, Columbia, SC (US); Tracy Marlatt, Lexington, SC (US); Samuel Edward Hartzog, Leesburg, GA (US); Scott Thoman, Columbia, SC (US); Travis Goldie, Lexington, SC (US)

(21) Appl. No.: 16/936,860

(22) Filed: Jul. 23, 2020

Related U.S. Application Data
(60) Provisional application No. 62/877,567, filed on Jul. 23, 2019.

(51) Int. Cl. G16H 50/20; G16H 70/20; G16H 50/70; G16H 40/20; G16H 10/60

(52) U.S. Cl. CPC G16H 50/20; G16H 70/20; G16H 10/60; G16H 40/20; G16H 50/70

(57) ABSTRACT

Systems and methods for training and deploying machine-learned models to predict medical codes are provided. In one example embodiment, a computing system can train machine-learned model(s) to predict medical codes based on training medical data. The machine-learned models can then be utilized to predict medical codes based on medical data. This can include a two stage model architecture. The two-stage architecture can include, for example, a unbiased machine-learned model trained to predict medical codes and a biased machine-learned model trained to revise the predicted medical codes according to a specific domain.

### PRIORITY CLAIM

The application is based on and claims benefit of United States Provisional Application 62/877,567 having a filing date of Jul. 23, 2019, which is incorporated in its entirety by reference herein.

### FIELD

The present disclosure relates generally to systems and methods for predicting identifiers for input features using a machine-learned model. More particularly, the systems and methods described herein can train the machine-learned model to simulate the decision-making capability of a medical expert by predicting identifiers for medical related input features.

### BACKGROUND

Information workers spend 25% of their time doing repetitive digital tasks. For some industries, such as medical coding, this repetitiveness approaches 90%. Medical coding is the process of translating a medical record into standardized codes which health insurance companies require to calculate provider reimbursement.

### SUMMARY

Aspects and advantages of embodiments of the present disclosure will be set forth in part in the following description, or may be learned from the description, or may be learned through practice of the embodiments.

One example aspect of the present disclosure is directed to a computer-implemented method. The method includes providing, by a computing system including one or more computing devices, a first set of input data to an unbiased machine-learned model. The input data includes one or more input features indicative of medical data. The method includes determining, by the computing system using the unbiased machine-learned model, one or more predicted identifiers based at least in part on the first set of input data. The predicted identifiers include one or more medical codes associated with the medical data. The method includes providing, by the computing system, a second set of input data to a biased machine-learned model. The second set of input data includes the one or more predicted identifiers and the medical data. The method includes receiving, by the computing system as an output of the biased machine-learned model, one or more revised predicted identifiers. The one or more revised predicted identifiers can include one or more different medical codes associated with the medical data descriptions.

Another example aspect of the present disclosure is directed to a computing system. The computing system includes a first machine-learned model configured to determine one or more predicted medical codes. The computing system includes a second machine-learned model configured to determine one or more revised predicted medical codes. The computing system includes one or more processors and one or more memories including instructions that, when executed by the one or more processors, cause the one or more processors to perform operations. The operations include providing input data to the first machine-learned model. The input data include one or more input features associated with medical data. The operations include receiving, as an output of the first machine-learned model, data indicative of the one or more predicted medical codes associated with the medical data. The operations include providing the output of the first machine-learned model as an input to the second machine-learned model. The operations include receiving, as an output of the second machine-learned model, a confirmation of the one or more predicted medical codes or the one or more revised predicted medical codes.

Yet another example aspect of the present disclosure is directed to a computer-implemented method for model training. The method includes receiving, by a computing system including one or more computing devices, training data. The training data includes one or more training features and one or more training identifiers, the one or more training features being associated with training medical data and the one or more training identifiers include one or more training medical codes. The method includes training, by the computing system, a machine-learned model based at least in part on the one or more training features and the one or more training identifiers. The method includes providing, by the computing system, input data to the machine-learned model, the input data including medical data. The method includes receiving, by the computing system as an output of the machine-learned model, data indicative of one or more predicted identifiers. The one or more predicted identifiers include one or more predicted medical codes based at least in part on the medical data. The method includes providing, by the computing system, the one or more predicted identifiers to a user device. The method includes receiving, by the computing system via the user device, feedback data associated with the one or more predicted identifiers. The feedback data includes an indication that the one or more predicted medical codes are correct or incorrect. The method includes re-training, by the computing system, the machine-learned model based at least in part on the feedback data.

Other example aspects of the present disclosure are directed to systems, methods, apparatuses, tangible, non-transitory computer-readable media, user interfaces, memory devices, and user devices for predicting identifiers for input features using a machine-learned model (e.g., in the medical related field, etc.).

These and other features, aspects, and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE DRAWINGS

Detailed discussion of embodiments directed to one of ordinary skill in the art are set forth in the specification, which makes reference to the appended figures, in which:

FIG. 1A depicts an example system architecture according to example embodiments of the present disclosure;

FIG. 1B depicts example machine-learned models according to example embodiments of the present disclosure;

FIG. 1C depicts an example semantic graph according to example embodiments of the present disclosure;

FIG. 2A depicts an example method according to example embodiments of the present disclosure;

FIG. 2B depicts an example method according to example embodiments of the present disclosure;

FIG. 3 depicts example computing system components according to example embodiments of the present disclosure;

FIG. 4 depicts an example user interface according to example embodiments of the present disclosure;

FIG. 5 depicts an example user interface according to example embodiments of the present disclosure;

FIG. 6 depicts an example user interface according to example embodiments of the present disclosure; and

FIG. 7 illustrates an example data flow diagram according to example embodiments of the present disclosure.

### DETAILED DESCRIPTION

Reference now will be made in detail to embodiments, one or more example(s) of which are illustrated in the drawings. Each example is provided by way of explanation of the embodiments, not limitation of the present disclosure. In fact, it will be apparent to those skilled in the art that various modifications and variations can be made to the embodiments without departing from the scope or spirit of the present disclosure. For instance, features illustrated or described as part of one embodiment can be used with another embodiment to yield a still further embodiment. Thus, it is intended that aspects of the present disclosure cover such modifications and variations.

Example aspects of the present disclosure are directed generally to systems and methods for training a machine-learned model. The machine-learned model can be configured to predict one or more identifiers for input data including one or more features. Training the machine-learned model can refer to modifying the machine-learned model based on performance on a training data set to improve the accuracy of a prediction from the machine-learned model, such as the predicted one or more identifiers.

One approach to training a machine-learned model includes providing a set of training data including one or more training features and one or more training identifiers. The model can learn relationships between the training features and training identifiers and extrapolate those relationships to predict features for new identifiers. However, the predictions from machine-learned models can, in some cases, be incorrect. It can be desirable to improve the accuracy of the machine-learned model to reduce a number of incorrect predictions. Additionally, it can be desirable to increase a confidence associated with the predictions.

According to example aspects of the present disclosure, feedback from a user based on a prediction from the machine-learned model can be provided as new training data to the machine-learned model. By providing manual feedback to the machine-learned model, it is possible to improve the accuracy of the machine-learned model in future prediction cases.

According to example aspects of the present disclosure, features can refer to any relevant input to a machine-learned model. For instance, features can include data associated with one or more identifiers. For instance, in cases including linguistic data, features can include key words or phrases within a document, excerpt, sentence, or other suitable grouping of text. For instance, features can include technical jargon or selectively important terms. As an example, features can refer to medical data such as diagnoses, medical records, prescriptions, or any other suitable medical data and, more particularly, excerpts or samples of the medical data. Additionally, or alternatively, features can refer to symptomatic language in the medical data, including occurrences of words such as, for example, “GI bleeding,” “anemia,” “blood loss,” and/or any other similar words. The features can be represented in any suitable format, such as textual and/or audial. The features can be converted to a computer-readable format, such as Unicode or ASCII, in accordance with example aspects of the present disclosure.

According to example aspects of the present disclosure, identifiers can refer to any data that can be associated with features. For instance, identifiers can have a dependent relationship with the features, such that a particular type and/or value of identifier depends on the type of feature with which the identifier is associated. As one example, identifiers can include medical codes that can be associated with medical data. For instance, the medical codes can concisely represent a symptom, setting, treatment, or other suitable aspect of medical care. Example medical codes include ICD (International Classification of Diseases) codes and/or other types of code.

The systems and methods described herein provide a number of technical effects and benefits. More particularly, the systems and methods described herein can utilize a machine-learned model to determine identifiers (e.g., medical codes, etc.) without the computational load and timing required by traditional searching. Moreover, the machine-learned model can be automatically trained and re-trained over time based on identifiers, feedback data, etc. to improve its accuracy. As such, the systems and methods of the present disclosure are more computationally flexible and provide an advantage over rules-based detection systems that require manual adjustment to improve its rules over time.

The systems and methods of the present disclosure provide an improvement to computing technology such as, for example, acoustic detection computing technology. The systems and methods of the present disclosure enable a computing system to determine an identifier (e.g., medical codes, etc.) given certain features (e.g., input data related to symptoms, etc.) and improve model accuracy over time. For example, a computing system can obtain input data from a source (e.g., textual/audible data from medical personnel, etc.). The computing system can access data indicative of a machine-learned model. The computing system can feed the input data from the source into the machine-learned model which can extract certain features (e.g., symptomatic information, etc.) and can obtain an output from the machine-learned model. The output can be indicative of an identifier associated with the features. For example, the identifier can be a medical code associated with the extract symptomatic information. The computing system can provide the output data to a user device of a user. The user can then utilize the output (e.g., medical code, etc.) and/or provide feedback, which can provide a basis for a reinforcement learning approach for the machine-learned model. In this way, the systems and methods of the present disclosure can leverage input data (e.g., medical data, etc.) and user feedback to automatically re-train its models to improve model accuracy for identifier predictions.

For the systems and methods of the present disclosure, a user can be provided with controls allowing the user to make an election as to both if and when systems, programs, or features described herein may enable collection of information (e.g., medical data, etc.), and if such information can be used for aggregate data purposes (e.g., to generate hierarchies, train models, etc.). In addition, certain data may be treated in one or more ways before it is stored or used, so that personally identifiable information is removed. For example, a user's/source's identity may be treated so that no personally identifiable information can be determined for the user/source. Thus, a user may have control over what information is collected and how that information is used.

Moreover, the systems and methods of the present disclosure can include a combination of trained machine-learned models to more accurately tailor the intended model output. For example, as further described herein, a first machine-learned model (e.g., graph network, etc.) can provide an unbiased output that indicates medical codes for medical conditions/services performed based at least in part on the relationships between the terms and symptoms appearing in the medical records and the assigned code as determine by a semantic graph. The output of the first unbiased machine-learned model can be utilized as an input into a second machine-learned model. The second machine-learned model can be domain biased in that it can be tailored based on certain characteristics associated with the input data (e.g., the hospital where it was created, the medical field, the provider group, etc.) and/or the ultimate consumer of the outputted date (e.g., the use of the medical code assignments, etc.). Accordingly, the systems and methods of the present disclosure can utilize this two stage unbiased/biased model approach to efficiently achieve results that can be effectively (and automatically) customized based on the circumstances.

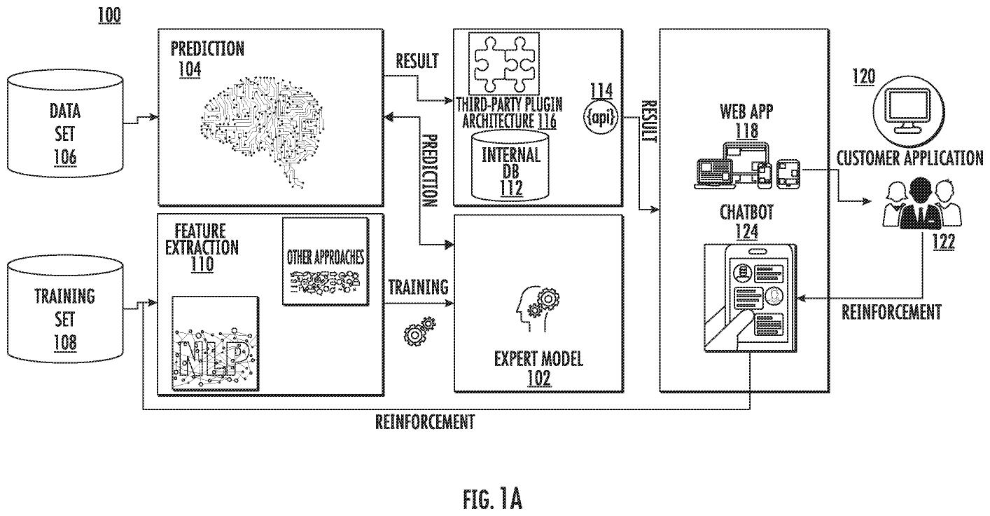

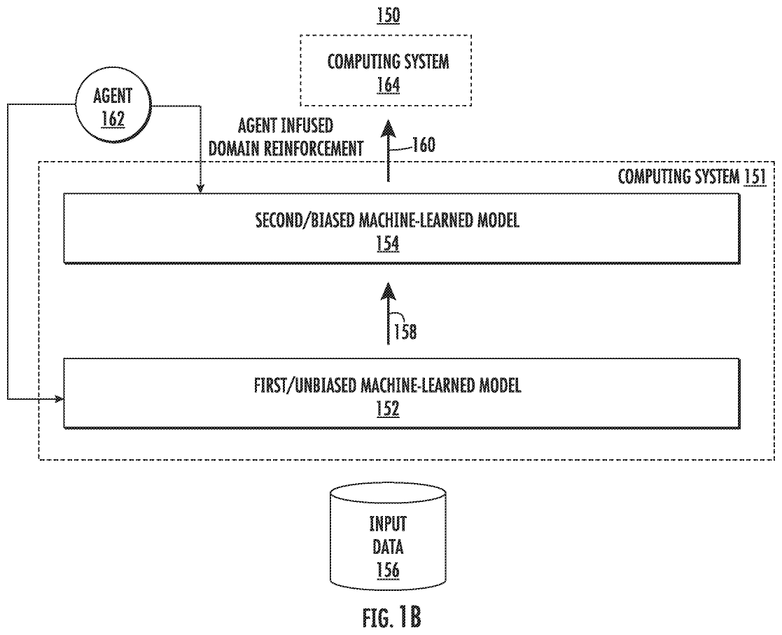

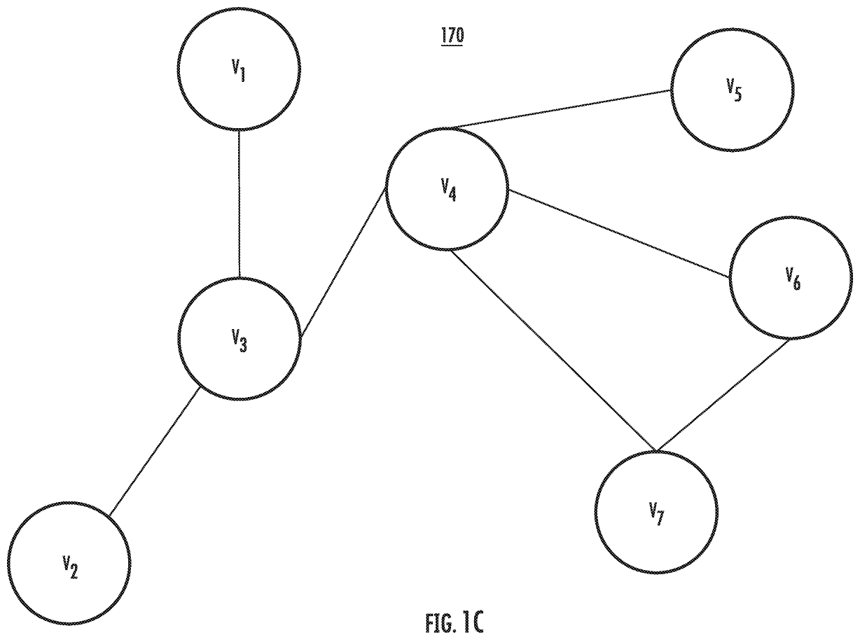

With reference now to the FIGS., example embodiments of the present disclosure will be discussed in further detail. FIG. 1A depicts an example system 100 according to example embodiments of the present disclosure. The system 100 can include an expert model 102. The expert model 102 can be configured to receive input data from data set 106. For instance, the input data can include one or more input features. The expert model 102 can output a prediction 104 based on the input data from data set 106. For instance, the model 102 can predict identifiers associated with the input features.

One example expert model 102 can include a term frequency-inverse document frequency (TF-IDF) model. TF-IDF can represent an importance or weighting of a word as a measure of how frequently the word appears in a larger collection of words (e.g., a document, etc.). In some cases the frequency of how often a word appears in a document is offset by how frequently the word appears in a collection of documents, such that words common to many documents (e.g., “the,” etc.) are not improperly assigned high important to a document. TF-IDF can be effective for learning an importance of a word.

Another example expert model 102 can be a semantic graph model. In a semantic graph model, features and identifiers are represented as nodes, and relationships between features and identifiers are represented as edges between corresponding nodes. In some cases, the edges are directed to indicate a one-way relationship. A variation of a semantic graph, called a semantic weighted graph, can include probabilistic weightings associated with each of the edges. The probabilistic weights can represent a distance between the nodes. The distance between the nodes can be indicative of a “closeness” of the concepts associated with the nodes. For instance, a short distance between a feature node and an identifier node can indicate that the feature and identifier are closely related.

Semantic graphs can allow for relatively quick prediction times. For instance, input data can be represented as an overlay over a semantic graph created from training data. A comparison of the overlay to the semantic graph can indicate similarities between the input data and training data, which can be useful in predicting the corresponding output for the input data.

In some embodiments, the semantic graphs can include an uncertainty associated with some or all of the nodes and/or edges. The uncertainty can be associated, for example, with a confidence score of a prediction based on the node and/or edge. For instance, the uncertainty can be provided by a combination of the semantic graph with one or more additional models.

The system 100 can include a training set 108. The training set 108 can include, for example, one or more training examples. For instance, each of the training examples can include one or more features and/or one or more identifiers. For example, in cases where the training set includes medical data, the training examples can be medical data having symptom descriptions and assigned medical codes.

In some cases, the training set 108 can be provided directly to train the expert model 102. For instance, training the model 102 can refer to modifying some aspect of the model 102, such as one or more parameters defining the model 102, based on the training set 108. For example, the model 102 can be adjusted based on the output of an objective function.

In some cases, the training set 108, before being provided to the expert model 102, can be provided to feature extraction 110 to extract one or more training features and/or one or more training identifiers. Any suitable method of feature extraction 110 can be utilized in accordance with the present disclosure. For instance, in embodiments where the data set 106 and/or the training set 108 includes linguistic data, the feature extraction 110 can extract features and/or identifiers to be used with natural language processing methods. For example, in some embodiments the feature extraction 110 can extract industry-specific terms or jargon from the training set 108 and/or training examples.

The expert model 102 can be configured to provide a prediction 104. For instance, the expert model 102 can provide a prediction 104 based on the results of being trained with training set 108. For example, the expert model 102 can learn relationships between features and identifiers as a result of being trained with training set 108. The expert model 102 can then receive input data (e.g. from data set 106, etc.) including input features and provide a prediction 104 of identifiers based on the input features. For example, in cases where the data set 106 and/or the training set 108 includes medical data, the input data can include medical data without assigned medical codes, and the prediction 104 can include predicted medical codes for the medical data.

The prediction 104 can be provided to various modules within the system 100. For instance, the prediction 104 can be stored in an internal database 112. As another example, the prediction 104 can be provided to an advanced programming interface (API) 114. From the API 114, the prediction can be provided to external programs accessing the API, such as web app 118. Additionally and/or alternatively, the prediction 104 can be provided to a plugin architecture 116. For example, the plugin architecture 116 can be configured to provide the prediction 104 to an external application 120, such as a client application.

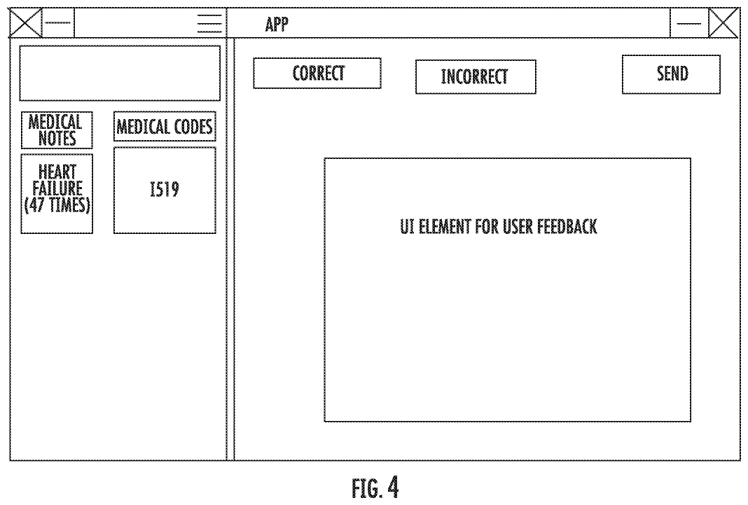

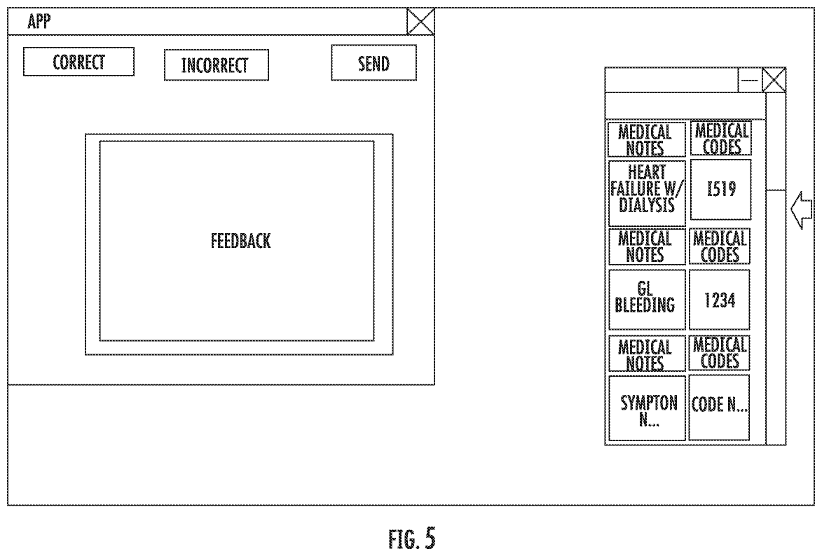

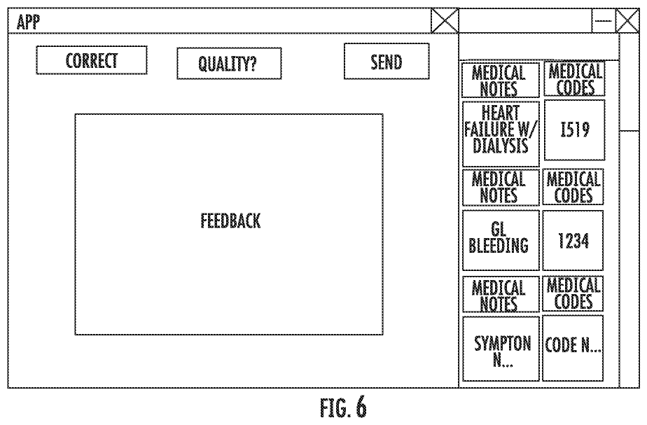

Example implementations of the web app 118 according to example aspects of the present disclosure are illustrated in FIGS. 4-6. These example user interfaces can display the prediction 104 of expert model 102. For example, the computing system 100 can provide, for display via one or more of the user interfaces of FIGS. 4-6, the predicted medical code as predicted from the extracted features of the data set (e.g., medical information, symptomatic information, etc.). The web app 118 can be configured to provide data to a user 122. For instance, the web app 118 can provide the prediction 104 to the user 122. Additionally and/or alternatively, the web app 118 can provide some or all of the input data from data set 106 to the user 122. For example, the web app 118 can provide the prediction 104 along with any input data that influenced the prediction 104. As another example, the web app 118 can indicate a correlation between input features in the input data and the predicted indicators in the prediction 104.

Additionally, the system 100 can include a feedback interface 124. The feedback interface 124 can be configured to receive feedback from the user 122 on the prediction 104 (e.g., predicted medical code, etc.) and provide the feedback to train the expert model 102. For example, the feedback interface 124 can provide the feedback as training data to feature extraction 110. For instance, an example feedback interface 124 can be a “ChatBot” that resembles a human messaging service.

FIG. 1B depicts example machine-learned models 150 according to example embodiments of the present disclosure. The machine-learned models 150 can represent an example architecture of the expert model 102. The model architecture can include a two stage architecture. The machine-learned models 150 can include a first machine-learned model 152 and a second machine-learned model 154. A computing system 151 can implement, access, and/or otherwise utilize the first and second machine-learned models 152, 154.

The first machine-learned model 152 can be configured to determine one or more predicted identifies such as, for example, one or more predicted medical codes. The first machine-learned model 152 can be a “unbiased” machine-learned model. The unbiased machine-learned model can determine predicted identifier(s) based on how it was trained and the data on which it was trained. The unbiased machine-learned model may not be trained, for example, to take into account the specific nuances, characteristics, circumstances, or practices of a domain.

The first machine-learned model can be configured to associate the one or more medical codes with the medical data based at least in part on a semantic graph such as, for example, the semantic graph 170 of FIG. 1C. The semantic graph 170 can include a plurality of vertices/nodes (V₁₋₇ . . . ) and edges between the vertices/nodes. As described herein, input features and identifiers can be represented by the vertices/nodes (e.g., “feature nodes,” “identifier nodes,” etc.), and relationships between the input features and identifiers can be represented as edges between corresponding nodes. For example, the vertices/nodes of the semantic can represent medical data and medical codes. The medical data can include symptom terms/phrase/descriptions, conditions, other medical terms, jargon, etc. An example of medical data can include “heart failure,” “dialysis,” “GI bleeding,” etc. As described herein, the medical codes can be a character, sequence of characters, value, etc. Example medical codes can include ICD (International Classification of Diseases) codes and/or another type of code. The medical codes can be those utilized for insurance and billing purposes.

In some cases, the edges of the semantic graph 170 can be directed to indicate a one-way relationship. Additionally, or alternatively, the semantic graph 170 can include a semantic weighted graph. The semantic weighted graph can include probabilistic weightings associated with each of the edges. The probabilistic weights can represent a distance between the vertices/nodes. The distance between the vertices/nodes can be indicative of a “closeness” of the concepts associated with the vertices/nodes. For instance, a short distance between a feature vertex/node and an identifier vertex/node can indicate that the feature and identifier are closely related.

Returning to FIG. 1B, the first machine-learned model 152 can apply deep learning techniques to traverse a semantic graph to help determine the appropriate identifier given a set of input features. The first machine-learned model 152 can be or can otherwise include one or more various model(s) such as, for example, neural networks (e.g., deep neural networks, etc.) or other multi-layer non-linear models. Neural networks can include convolutional neural networks, recurrent neural networks (e.g., long short-term memory recurrent neural networks, etc.), and/or other forms of neural networks.

The first machine-learned model 152 can be trained to predict identifiers such as, for example, medical codes based on input feature such as, for example, medical data and the information included therein. By way of example, a computing system 151 can obtain input data 156. The input data 156 can include medical data. For example, input data 156 can include one or more input features associated with medical data (e.g., symptom descriptions, terms, etc. in a medical record). The computing system 151 can provide the input data 156 to the first machine-learned model 152. The layers of the first machine-learned model 152 can be configured to determine a path through the semantic graph to predict an identifier (e.g., a medical code, etc.) given a set of input features (e.g., medical data, etc.). By way of example, the input data 156 can include descriptions from a medical record such as, for example, “the patient is experiencing heart failure” (appearing several time) and “the patient is on dialysis.” The first machine-learned model 152 can utilize the semantic graph (e.g., its node/edges, etc.) to determine that the medical code “I519” is associated with “heart failure.”

The first machine-learned model 152 can generate an output 158 that includes data indicative of the one or more medical codes associated with the medical data. The computing system 151 can receive the data indicative of the one or more medical codes associated with the medical data as the output 158 of the first machine-learned model 152. The computing system 151 can provide the output 158 (or a processed version thereof) of the first machine-learned model 152 as an input to the second machine-learned model 154.

The second machine-learned model 154 can be configured to confirm the predicted medical code(s) and/or determine one or more revised predicted medical codes. The second machine-learned model 154 can be a “biased” machine-learned model. The biased machine-learned model can be configured to confirm that the predicted medical code(s) (e.g., output from the first machine-learned model 152) are accurate/correction/likely/etc. and/or determine the one or more revised predicted identifiers based at least in part on a domain. The biased machine-learned model can be trained to confirm and/or revise the predicted identifiers based at least in part on the specific nuances, characteristics, circumstances, practices, etc. of a domain. The domain can include, for example, at least one of an entity associated with the medical data (e.g., a physician, group, medical field, hospital, etc. associated with the medical record) or a consumer of the one or more revised predicted identifiers (e.g., a hospital, billing entity, insurance company, etc.).

The second machine-learned model 154 can be or can otherwise include one or more various model(s) such as, for example, neural networks (e.g., deep neural networks, etc.), or other multi-layer non-linear models. Neural networks can include convolutional neural networks, recurrent neural networks (e.g., long short-term memory recurrent neural networks, etc.), and/or other forms of neural networks. The second machine-learned model 154 can be trained with a labelled training data that indicates specific medical code practices or behaviors of a domain. This can allow the second machine-learning model 154 to potentially apply different weight (e.g., within a semantic graph, etc.) based at least in part on the domain. The different weights can result in a revised medical code being associated with the medical data.

By way of example, the data input into the second machine-learned model can include a portion of a medical record indicating “the patient is experiencing heart failure” and that “the patient is on dialysis” as well as the output of the first machine-learned model 152 associating the medical code “I519” with “heart failure.” The second machine-learned model 154 can utilize its domain infused knowledge to understand that the particular hospital that generated the medical data (e.g., the corresponding medical record, etc.) or the hospital for which the ultimate output is to be provided utilizes a different medical code when patients experience medical issues while on dialysis. This can be reflected in the weights applied by the second machine-learned model 154. Thus, the second machine-learned model 154 can determine that a revised medical code “I520” is associated with the medical data. The computing system 151 can receive, as an output 160 of the second machine-learned model 154, the one or more revised predicted medical codes. The computing system 151 can provide this information to another computing system 164 (e.g., the computing system of the consumer of the medical codes, etc.).

In another example, the second machine-learned model 154 can determine that the predicted medical code(s) from determined by the first machine-learned model 152 are accurate. For instance, the second machine-learned model 154 can confirm that the appropriate medical code for “heart failure” is “I519” based at least in part on the practices of the particular field of medicine, hospital, geographic region, insurer, etc. The output 160 of the second machine-learned model 154 can confirm/indicate a confirmation of the predicted medical code(s). For example, the output could repeat those codes and/or provide an indication that the inputted codes are accurate, appropriate, likely, etc. Thus, the computing system 151 can receive, as an output of the second machine-learned model 154, a confirmation of the one or more predicted medical codes or the one or more revised predicted medical codes.

The first machine-learned model 152 and the second machine-learned model 154 can be trained based at least in part on reinforcement learning. For instance, outputs of the first machine-learned model 152 and the second machine-learned model 154 (as well as corresponding input data) can be provided to a user device of an agent 162. The agent 162 can be a user that reviews the outputs via the user device and provides feedback including an indication that the one or more predicted medical codes and/or revised predicted medical codes are correct or incorrect. The user device can provide feedback data to the computing system 151 (and/or another training computing system) which can be used to re-train the first and/or second machine-learned models 152, 154, as described herein. In some implementations, the user/agent can provide additional information including, for example, whether it appears that the care was quality or not.

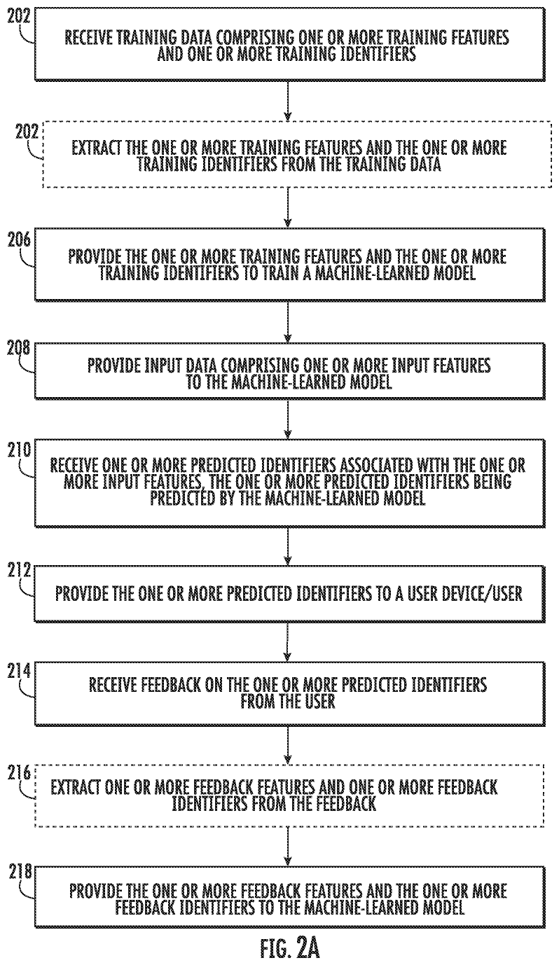

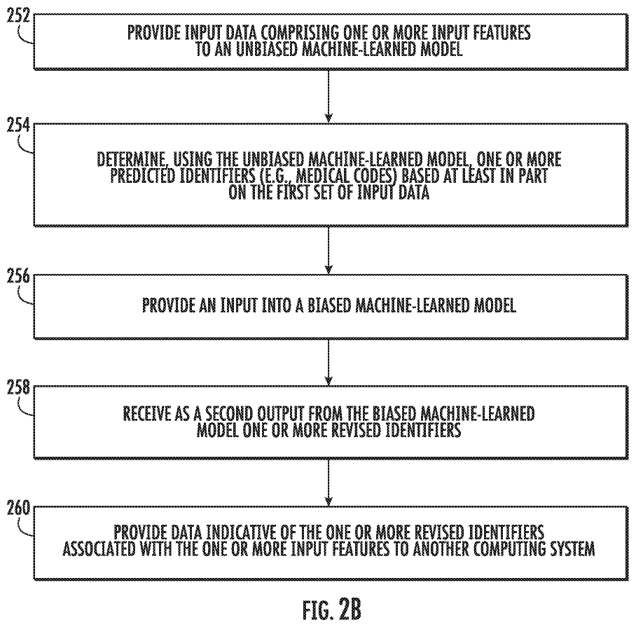

FIG. 2A depicts a flow diagram of an example method 200 of training a model for predicting identifiers associated with features according to example embodiments of the present disclosure. For example, the example method 200 can be utilized for predicting medical codes associated with medical data. One or more portion(s) of method 200 can be implemented by one or more computing device(s) such as, for example, those shown in FIGS. 1A-C and 3. Moreover, one or more portion(s) of the method 200 can be implemented as an algorithm on the hardware components of the device(s) described herein (e.g., as in FIGS. 1 and 2, etc.) to, for example, predict identifiers associated with input features. FIG. 2A depicts steps performed in a particular order for purposes of illustration and discussion. Those of ordinary skill in the art, using the disclosures provided herein, will understand that the steps of any of the methods discussed herein can be adapted, rearranged, expanded, omitted, or modified in various ways without deviating from the scope of the present disclosure.

At (202), the method 200 can include receiving training data. The training data can include one or more training features. The training data can include one or more training identifiers. In some cases, the training data can include one or more pieces of training data. For instance, each piece of training data can include one or more features associated with the piece of training data and one or more identifiers associated with the piece of training data.

By way of example, the training data can include medical data. The medical data can include a symptom description having one or more features (e.g., words and/or phrases describing symptoms, etc.) and/or one or more identifiers (e.g., medical codes, etc.). For example, in one example, the medical data can be of the form “‘The patient is experiencing GI bleeding’: 1234.” In another example, the medical data includes the symptom description “The patient is experiencing GI bleeding” and the identifier “1234.”

In some cases, the one or more training features and/or the one or more training identifiers can be extracted from the training data at (204). For instance, in some cases, the training data can include superfluous data, such as words or phrases that do not generally contribute to a correlation between the training data and the training identifiers. The one or more training features and/or the one or more training identifiers can thus be extracted from the training data to provide meaningful data to train the machine-learned model. For instance, in cases where the training data includes one or more medical data, training features such as symptoms (e.g., “GI bleeding,” etc.) and/or training identifiers such as medical codes (e.g. “1234,” etc.) can be extracted from the medical data (e.g. “‘The patient is experiencing GI bleeding’: 1234,” etc.).

At (206), the method 200 can include providing the one or more training features and/or the one or more training identifiers to train the machine-learned model. The machine-learned model can be trained in any suitable method in accordance with example aspects of the present disclosure. For instance, the machine-learned model can be trained by optimizing an objective function. As an example, the machine-learned model can be trained by adjusting one or more parameters of the machine-learned model.

At (208), the method 200 can include providing input data to the machine-learned model. The input data can include one or more input features. For instance, the one or more input features can be substantively similar to the training features. For example, in cases where the training data includes medical data, the input data can include a medical data. For example, the input data can include a medical data without identifiers, such a symptom description without assigned medical code(s).

At (210), the method 200 can include receiving one or more predicted identifiers associated with the input data. For instance, the predicted identifiers can be predicted by the machine-learned model. The predicted identifiers can be substantively similar to the training identifiers. For example, in cases where the training data includes medical data, the predicted identifiers can be medical codes associated with the input medical data.

At (212), the method 200 can include providing the one or more predicted identifiers to a user device/user. For instance, the one or more predicted identifiers can be provided to a user device of the user along with some or all of the input data. For example, the input features can be provided to the user along with the predicted identifiers associated with the input features. Additionally, in some embodiments, correlation between the predicted identifiers and the input features can be indicated.

At (214), the method 200 can include receiving feedback on the one or more predicted identifiers from the user. For instance, the user can provide an indication that the one or more predicted identifiers are incorrect. Additionally and/or alternatively, the user can provide one or more corrected identifiers as feedback. For instance, the one or more corrected identifiers can include identifiers (e.g., medical codes, etc.) that correctly correspond to the one or more input features (e.g., symptom descriptions, etc.) and/or the input data, such as in cases where the one or more predicted identifiers fail to correspond to the one or more input features and/or the input data. As another example, the user can provide an indication of what the one or more corrected identifiers correspond to. For example, the user can highlight or otherwise select (e.g., manually select, etc.) input features that correspond to the corrected identifiers.

Additionally and/or alternatively, the user can provide as feedback an explanation of why the one or more predicted identifiers are incorrect. For instance, in some cases the user can write or type a response explaining why the one or more predicted identifiers are incorrect. In some cases, the response can be phrased in a manner that resembles responding to a human. For instance, the response can be phrased conversationally. By way of example, this can include providing an explanation as to why the medical code is incorrect.

In some cases, one or more feedback features and/or one or more feedback identifiers can be extracted from the feedback at (216). For instance, in cases where a response indicating why the one or more predicted identifiers are incorrect is provided as feedback, the method 200 can include extracting one or more feedback features and/or one or more feedback identifiers from the response.

At (218), the method 200 can include providing feedback data including the one or more feedback features and/or the one or more feedback identifiers to the machine-learned model. For instance, the feedback data can be provided to train the machine-learned model. For instance, the feedback data can be provided to the machine-learned model in a similar manner to the training data. In some embodiments, the feedback data may be provided to the machine-learned model in a different manner than the training data. For instance, the feedback data may be weighted heavier than the training data, such that the feedback data has more of an effect on the machine-learned model than the training data. As another example, the feedback data may be treated as a truth or rule.

FIG. 2B depicts a flow diagram of an example method 250 of predicting identifiers (e.g., medical codes, etc.) according to example embodiments of the present disclosure. For example, the example method 250 can be utilized for predicting medical codes associated with medical data. One or more portion(s) of method 250 can be implemented by one or more computing device(s) such as, for example, those shown in FIGS. 1A-C and 3. Moreover, one or more portion(s) of the method 250 can be implemented as an algorithm on the hardware components of the device(s) described herein (e.g., as in FIGS. 1A-C and 3, etc.) to, for example, predict identifiers (e.g., medical codes, etc.) associated with input features (e.g., medical data, etc.). FIG. 2B depicts steps performed in a particular order for purposes of illustration and discussion. Those of ordinary skill in the art, using the disclosures provided herein, will understand that the steps of any of the methods discussed herein can be adapted, rearranged, expanded, omitted, or modified in various ways without deviating from the scope of the present disclosure.

At (252), the method can include providing input data comprising one or more input features to an unbiased machine-learned model (e.g. first machine-learned model 152, etc.). For example, a computing system can provide a first set of input data to an unbiased machine-learned model. The input data can include one or more input features indicative of medical data. As described herein, the unbiased machine-learned model can be configured to associate the one or more medical codes with the medical data based at least in part on a semantic graph.

At (254), the method can include determining one or more predicted identifiers based at least in part on the first set of input data. For example, the computing system can determine, using the unbiased machine-learned model, one or more predicted identifiers based at least in part on the first set of input data. The predicted identifiers comprise one or more medical codes associated with the medical data. The computing system, an output of the unbiased machine-learned model, the one or more predicted identifiers (e.g., medical codes, etc.).

At (256), the method can include an input into a biased machine-learned model (e.g., a second machine-learned model 154, etc.). For example, the computing system can provide a second set of input data to a biased machine-learned model. The second set of input data can include the one or more predicted identifiers (e.g., medical codes, etc.) and the medical data. The second set of input data can include the output of the unbiased machine-learned model. As described herein, the unbiased machine-learned model can be configured to determine the one or more revised predicted identifiers based at least in part on a domain. The domain can include, for example, at least one of an entity associated with the medical data or a consumer of the one or more revised predicted identifiers (e.g., hospital, insurance provider, etc.). The entity associated with the medical data can include at least one of a facility, a provider group, or a medical field.

At (258), the method can include receiving, as a second output from the biased machine-learned model, one or more revised identifiers. For example, the computing system can receive, as an output of the biased machine-learned model, one or more revised predicted identifiers. The one or more revised predicted identifiers can include one or more different medical codes associated with the medical data descriptions.

At (260), the method can include providing data indicative of the one or more revised identifiers associated with the one or more input features to another computing system. For example, the computing system can provide data indicative of the one or more revised predicted identifiers to another computing system. The other computing system can be associated with an entity that will utilize the revised identifiers (e.g., medical codes, etc.). The data indicative of the revised predicted identifiers can include the one or more different medical codes assigned to the medical data. As described herein, the medical data can be associated with one or more medical records.

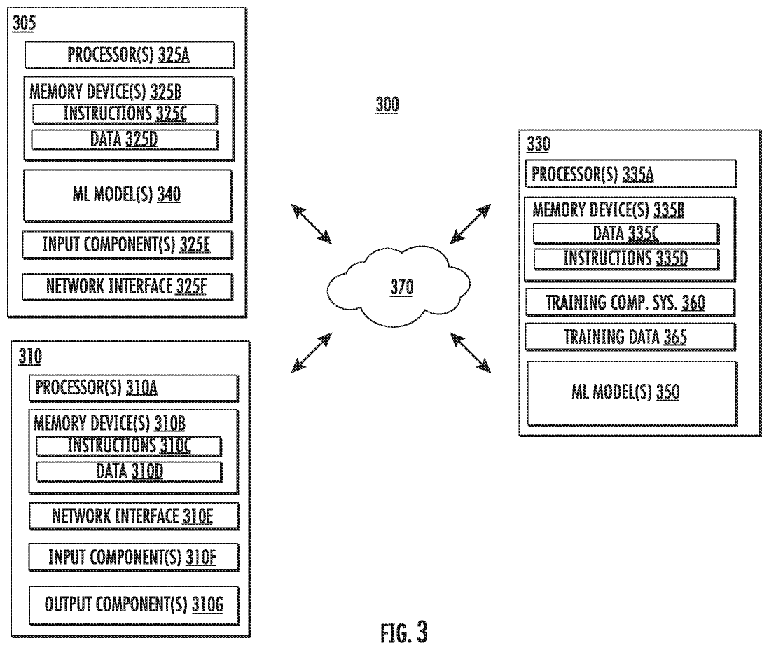

FIG. 3 depicts an example system 300 according to example embodiments of the present disclosure. The system 300 can include one or more user device(s) 310, the prediction computing system 305, and a machine learning computing system 330. One or more of these systems communicate over one or more network(s) 370.

A user device 310 can include one or more processor(s) 310A and one or more memory device(s) 310B. The one or more processor(s) 310A can include any suitable processing device, such as a microprocessor, microcontroller, integrated circuit, an application specific integrated circuit (ASIC), a digital signal processor (DSP), a field-programmable gate array (FPGA), logic device, one or more central processing units (CPUs), graphics processing units (GPUs), processing units performing other specialized calculations, etc. The memory device(s) 310B can include memory such as one or more non-transitory computer-readable storage medium(s), such as RAM, ROM, EEPROM, EPROM, flash memory devices, magnetic disks, etc., and/or combinations thereof.

The memory device(s) 310B can include one or more computer-readable media and can store information accessible by the one or more processor(s) 310A, including instructions 310C that can be executed by the one or more processor(s) 310A. For instance, the memory device(s) 310B can store instructions 310C for running one or more software applications, displaying a user interface, receiving user input, processing user input, playing audio data, etc. as described herein. In some embodiments, the instructions 310C can be executed by the one or more processor(s) 310A to cause the one or more processor(s) 310A to perform operations, such as any of the operations and functions for which the user device(s) 310 are configured, and/or any other operations or functions of the user device(s) 310, as described herein. The instructions 310C can be software written in any suitable programming language or can be implemented in hardware. Additionally, and/or alternatively, the instructions 310C can be executed in logically and/or virtually separate threads on processor(s) 310A.

The one or more memory device(s) 310B can also store data 310D that can be retrieved, manipulated, created, or stored by the one or more processor(s) 310A. The data 310D can include, for instance, data indicative of: output data, predicted identifiers, medical codes, user input, user interface(s), feedback data, etc. In some implementations, the data 310D can be received from another device.

The user device 310 can also include a network interface 310E used to communicate with one or more other component(s) of system 300 over the network(s) 370. The network interface 310E can include any suitable components for interfacing with one or more network(s), including for example, transmitters, receivers, ports, controllers, antennas, or other suitable components.

The user device(s) 310 can include one or more input component(s) 310F and/or one or more output component(s) 310G. The input component(s) 310F can include, for example, hardware and/or software for receiving information from a user, such as a touch screen, touch pad, mouse, data entry keys, speakers, a microphone, etc. The output component(s) 310G can include hardware and/or software for audibly producing audio data for a user. For instance, the audio output component 310G can include one or more speaker(s), earpiece(s), headset(s), handset(s), etc. The output component(s) 310G can include a display device, which can include hardware for displaying a user interface and/or messages for a user. By way of example, the output component 310G can include a display screen, CRT, LCD, plasma screen, touch screen, TV, projector, and/or other suitable display components.

The prediction computing system 305 can include one or more computing device(s) located at the same or different locations. The computing device(s) can include one or more processors 325A and a one or more memory devices 325B. The processor(s) 325A can be located at the same or different locations. Additionally, or alternatively, the memory device(s) 325B can be located at the same or different locations.

The one or more processors 325A can include any suitable processing device, such as a microprocessor, microcontroller, integrated circuit, an application specific integrated circuit (ASIC), a digital signal processor (DSP), a field-programmable gate array (FPGA), logic device, one or more central processing units (CPUs), graphics processing units (GPUs), processing units performing other specialized calculations, etc. The memory device(s) 325B can include memory such as one or more non-transitory computer-readable storage medium(s), such as RAM, ROM, EEPROM, EPROM, flash memory devices, magnetic disks, etc., and/or combinations thereof.

The memory device(s) 325B can include one or more computer-readable media and can store information accessible by the one or more processor(s) 325A, including instructions 325C that can be executed by the one or more processor(s) 325A. In some embodiments, the instructions 325C can be executed by the one or more processor(s) 325A to cause the one or more processor(s) 325A to perform operations, such as any of the operations and functions for which the prediction computing system 305 is configured, one or more operations and functions for predicting identifiers (e.g., medical codes, etc.) associated with features (e.g., one or more portions of method 200, etc.) and/or any other operations or functions of the prediction computing system 305, as described herein. The instructions 325C can be software written in any suitable programming language or can be implemented in hardware. Additionally, and/or alternatively, the instructions 325C can be executed in logically and/or virtually separate threads on processor(s) 325A.

The one or more memory device(s) 325B can also store data 325D that can be retrieved, manipulated, created, or stored by the one or more processor(s) 325A. The data 325D can include, for instance, data associated with: a data source, model(s), input data, features, predictions, identifiers, medical codes, feedback data, and/or any other data/information described herein. In some implementations, the data 325D can be received from another device.

The prediction computing system 305 can also include a network interface 325F used to communicate with one or more other component(s) of system 300 over the network(s) 330. The network interface 325F can include any suitable components for interfacing with one or more network(s), including for example, transmitters, receivers, ports, controllers, antennas, or other suitable components.

The computing device(s) of the prediction computing system 305 can include one or more input component(s) 325E. The input component(s) 325E can include, for example, hardware and/or software for receiving input data from a source such as, for example, a microphone and/or other audio content capturing technology, etc.

According to an aspect of the present disclosure, prediction system 305 can store and/or include one or more machine-learned models 340. As examples, the machine-learned models 340 can be or can otherwise include various machine-learned models such as, for example, neural networks (e.g., deep neural networks, etc.), support vector machines, decision trees, ensemble models, k-nearest neighbors models, Bayesian networks, or other types of models including linear models and/or non-linear models. Example neural networks include recurrent neural networks (e.g., long short-term memory recurrent neural networks, etc.), or other forms of neural networks. The machine-learned models 340 can include the one or more machine-learned models, as described herein with reference to FIGS. 1-3.

In some implementations, the prediction computing system 305 can receive the one or more machine-learned models 340 from the machine learning computing system 330 (e.g., a server computing system, etc.) over the network(s) 370 and can store the one or more machine-learned models 340 in the memory of the respective system. The machine learning computing system 330 can be a portion of and/or separate from the prediction system 305. The prediction system 305 can use or otherwise implement the one or more machine-learned models 340 (e.g., by processor(s) 325A, etc.).

The machine learning computing system 330 can include one or more processors 335A and memory device(s) 335B. The one or more processors 335A can be any suitable processing device (e.g., a processor core, a microprocessor, an ASIC, a FPGA, a controller, a microcontroller, etc.) and can be one processor or a plurality of processors that are operatively connected. The memory 335B can include one or more non-transitory computer-readable storage media, such as RAM, ROM, EEPROM, EPROM, one or more memory devices, flash memory devices, etc., and combinations thereof.

The memory device(s) 335B can store information that can be accessed by the one or more processors 335A. For instance, the memory device(s) 335B (e.g., one or more non-transitory computer-readable storage mediums, memory devices, etc.) can include one or more memory that can store data 335C that can be obtained, received, accessed, written, manipulated, created, and/or stored. In some implementations, the machine learning computing system 330 can obtain data from one or more memory devices that are remote from the machine learning computing system 330.

The memory device(s) 335B can also store computer-readable instructions 335D that can be executed by the one or more processors 335A. The instructions 335D can be software written in any suitable programming language or can be implemented in hardware. Additionally, or alternatively, the instructions 335D can be executed in logically and/or virtually separate threads on processor(s) 335A. The memory device(s) 335B can store the instructions 335D that when executed by the one or more processors 335A cause the one or more processors 335A to perform operations. The machine learning computing system 330 can include a communication interface, including devices and/or functions similar to that described with respect to the prediction computing system 305.

In some implementations, the machine learning computing system 330 can include one or more server computing devices. If the machine learning computing system 330 includes multiple server computing devices, such server computing devices can operate according to various computing architectures, including, for example, sequential computing architectures, parallel computing architectures, or some combination thereof.

In addition or alternatively to the machine-learned model(s) 340 at the prediction computing system 305, the machine learning computing system 330 can include one or more machine-learned models 350. As examples, the machine-learned model(s) 350 can be or can otherwise include various machine-learned models such as, for example, neural networks (e.g., deep neural networks, etc.), support vector machines, decision trees, ensemble models, k-nearest neighbors models, Bayesian networks, or other types of models including linear models and/or non-linear models. Example neural networks include recurrent neural networks (e.g., long short-term memory recurrent neural networks, or other forms of neural networks, etc.). The machine-learned models 350 can be similar to and/or the same as the machine-learned models 340. In some implementations, the machine-learned models described herein may not include a feed-forward network.

As an example, the machine learning computing system 330 can communicate with the prediction computing system 305 according to a client-server relationship. For example, the machine learning computing system 330 can implement the machine-learned models 350 to provide a web service to the prediction computing system 305. For example, the web service can provide machine-learned models to an entity associated with the prediction computing system 305; such that the entity can implement the machine-learned model (e.g., to predict medical codes, etc.). Thus, machine-learned models 350 can be located and used at the prediction computing system 305 and/or machine-learned models 350 can be located and used at the machine learning computing system 330.

In some implementations, the machine learning computing system 330 and/or the prediction computing system 305 can train the machine-learned models 340 and/or 350 through the use of a training computing system 360. The training computing system 360 can include one or more processors and a memory similar to those described herein for the other components of the system 300. The memory can store information that can be accessed by the one or more processors. For instance, the memory (e.g., one or more non-transitory computer-readable storage mediums, memory devices, etc.) can store data that can be obtained, received, accessed, written, manipulated, created, and/or stored. The memory can store the instructions that when executed by the one or more processors cause the one or more processors to perform operations.

The training computing system 360 can include a model trainer. The model trainer can train the machine-learned models 340 and/or 350 using one or more training or learning algorithms. One example training technique is backwards propagation of errors. In some implementations, the model trainer can perform supervised training techniques using a set of labeled training data. In other implementations, the model trainer can perform unsupervised training techniques using a set of unlabeled training data. The model trainer can perform a number of generalization techniques to improve the generalization capability of the models being trained. Generalization techniques can include, for example, weight decays, dropouts, or other techniques. As described herein, the model trainer can utilize reinforced learning techniques. In particular, the model trainer can train a machine-learned model 340 and/or 350 based on a set of training data 365. The training data 365 can include, for example, the training data as described herein. The model trainer can be implemented in hardware, firmware, and/or software controlling one or more processors. The training computing system 360 (and/or model trainer) can be included in and/or separate from the machine learning computing system 330.

In some cases, the training computing system 360 and/or the training data 365 may be processed in an isolated location away from the location of the machine-learned models 340 and/or 350 (e.g., the models described herein, etc.). In such cases, a machine learning algorithm and feature extraction algorithm may be sent to the isolated location in order to process the training set and update the machine-learned models 340 and/or 350 without the removal of the training data 365 from the isolated location. The training can involve a federated learning process.

The network(s) 370 can be any type of network or combination of networks that allows for communication between devices. In some embodiments, the network(s) 370 can include one or more of a local area network, wide area network, the Internet, secure network, cellular network, mesh network, peer-to-peer communication link and/or some combination thereof and can include any number of wired or wireless links. Communication over the network(s) 370 can be accomplished, for instance, via a network interface using any type of protocol, protection scheme, encoding, format, packaging, etc.

FIG. 3 illustrates one example system 300 that can be used to implement the present disclosure. Other computing systems can be used as well. For example, in some implementations, the prediction computing system 305 can include the training computing system 360, model trainer, and/or the set of training data 365. In such implementations, the machine-learned models 340 can be both trained and used locally at the prediction computing system 305. As another example, in some implementations, the prediction computing system 305 may not be connected to other computing systems.

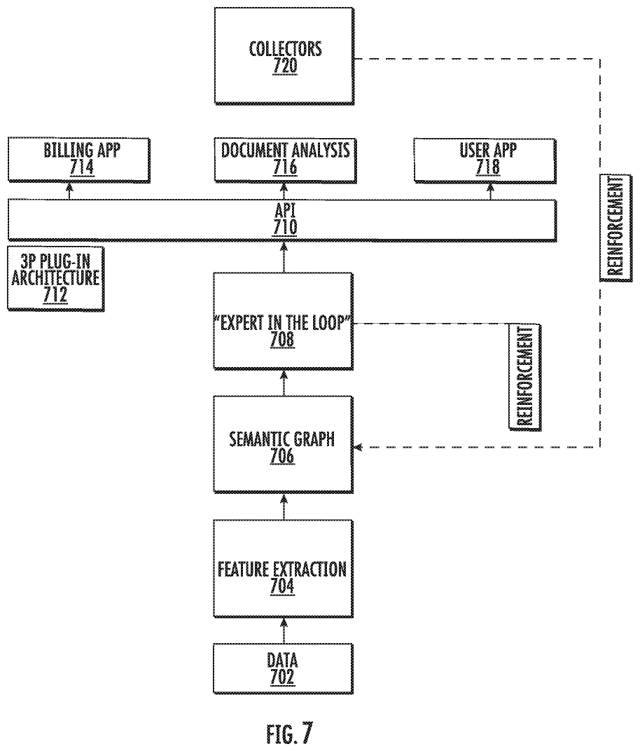

FIG. 7 illustrates an example data flow diagram according to example embodiments of the present disclosure. As described herein, data 702 can be ingested in a computing system (e.g., the systems of FIGS. 1A & 1B). The data 702 can be associated with medical data. For instance, the data 702 can be data/information from medical records. Input features 704 can be extracted. The input features 704 can include symptoms and/or other descriptors associated with a patient being evaluated and recorded in a medical record. The computing system can extract the features 704 from the data 702 for analysis via the model(s) described herein. The semantic graph 706 can include a deep semantic graph. The semantic graph 706 can include a scalable ontology. As described herein, the model(s) utilizing the semantic graph 706 can employ reinforced learning. Additionally, or alternatively, the semantic graph 706 can utilize sub-graph training techniques for training at the edge.

The model(s) utilizing the semantic graph 706 can provide measurable confidence and uncertainty. For example, the model(s) can provide an uncertainty score and/or a confidence level associated with the one or more predicted (and/or revised predicted) identifiers as an output. The uncertainty score and/or confidence level can indicate how confident the model(s) are with respect to the predicted/revised predicted identifiers (e.g., the higher the confidence level, the higher the confidence; the higher the uncertainty score, the higher the uncertainty, etc.). In the event the output is associated with a low uncertainty score/confidence level (e.g., below 90, 80, 70, 50 percent, etc.), an expert in the loop 708 can be utilized to evaluate the predicted/revised predicted identifiers (e.g., medical codes).

A plugin architecture 712 can be configured to provide access to the model output(s) and the model(s) described herein. For example, one or more APIs 710 can be utilized for facilitating communications between the system implementing the model(s) described herein and one or more other applications. The other applications can include client applications such as a billing application 714 (e.g., for automated medical insurance and billing purposes based on the predicted medical codes), a document analysis application 716 (e.g., for indicating the number/patterns of terms and descriptors within inputted data, predicted identifiers associated therewith, etc.), a user application 718 (e.g., for acquiring feedback from a community of collectors 720, for reporting predicted identifiers to medical/insurance purposes, etc.), and/or other types of applications. Moreover, the community of collectors 720 can help facilitate the reinforcement training techniques described herein.

The technology discussed herein makes reference to servers, databases, software applications, and other computer-based systems, as well as actions taken and information sent to and from such systems. One of ordinary skill in the art will recognize that the inherent flexibility of computer-based systems allows for a great variety of possible configurations, combinations, and divisions of tasks and functionality between and among components. For instance, server processes discussed herein can be implemented using a single server or multiple servers working in combination. Databases and applications can be implemented on a single system or distributed across multiple systems. Distributed components can operate sequentially or in parallel.

Furthermore, computing tasks discussed herein as being performed at a server can instead be performed at a user device. Likewise, computing tasks discussed herein as being performed at the user device can instead be performed at the server.

While the present subject matter has been described in detail with respect to specific example embodiments and methods thereof, it will be appreciated that those skilled in the art, upon attaining an understanding of the foregoing can readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

What is claimed is:

1. A computer-implemented method comprising:
providing, by a computing system comprising one or more computing devices, a first set of input data to an unbiased machine-learned model, wherein the input data comprises one or more input features indicative of medical data;
determining, by the computing system using the unbiased machine-learned model, one or more predicted identifiers based at least in part on the first set of input data, wherein the predicted identifiers comprise one or more medical codes associated with the medical data;
providing, by the computing system, a second set of input data to a biased machine-learned model, the second set of input data comprising the one or more predicted identifiers and the medical data; and
receiving, by the computing system as an output of the biased machine-learned model, one or more revised predicted identifiers, wherein the one or more revised predicted identifiers comprises one or more different medical codes associated with the medical data descriptions.

2. The computer-implemented method of claim 1, wherein the unbiased machine-learned model is configured to associate the one or more medical codes with the medical data based at least in part on a semantic graph.

3. The computer-implemented method of claim 1, wherein the biased machine-learned model is configured to determine the one or more revised predicted identifiers based at least in part on a domain.

4. The computer-implemented method of claim 3, wherein the domain comprises at least one of an entity associated with the medical data or a consumer of the one or more revised predicted identifiers.

5. The computer-implemented method of claim 4, wherein the entity associated with the medical data comprises at least one of a facility, a provider group, or a medical field.

6. The computer-implemented method of claim 1, wherein the unbiased machine-learned model and the biased machine-learned model are trained based at least in part on reinforcement learning.

7. The computer-implemented method of claim 1, wherein determining, by the computing system using the unbiased machine-learned model, the one or more predicted identifiers based at least in part on the first set of input data comprises:
providing, by the computing system, the first set of input data to the unbiased machine-learned model; and
receiving, by the computing system as an output of the unbiased machine-learned model, the one or more predicted identifiers.

8. The computer-implemented method of claim 1, wherein the second set of input data comprises an output of the unbiased machine-learned model.

9. The computer-implemented method of claim 1, further comprising:
providing, by the computing system, data indicative of the one or more revised predicted identifiers to another computing system.

10. The computer-implemented method of claim 1, wherein the data indicative of the revised predicted identifiers comprises the one or more different medical codes assigned to the medical data.

11. The computer-implemented method of claim 10, wherein the medical data is associated with one or more medical records.

12. A computing system comprising:
a first machine-learned model configured to determine one or more predicted medical codes;
a second machine-learned model configured to determine one or more revised predicted medical codes;
one or more processors; and
one or more memories including instructions that, when executed by the one or more processors, cause the one or more processors to perform operations, the operations comprising:
providing input data to the first machine-learned model, wherein the input data comprises one or more input features associated with medical data;
receiving, as an output of the first machine-learned model, data indicative of the one or more predicted medical codes associated with the medical data;
providing the output of the first machine-learned model as an input to the second machine-learned model; and
receiving, as an output of the second machine-learned model, a confirmation of the one or more predicted medical codes or the one or more revised predicted medical codes.

13. The computing system of claim 12, wherein the first machine-learned model is configured to associate the one or more predicted medical codes with the medical data based at least in part on a semantic graph.

14. The computing system of claim 12, wherein the second machine-learned model is configured to determine the one or more revised predicted identifiers based at least in part on a domain.

15. The computing system of claim 12, wherein the domain comprises at least one of an entity associated with the medical data or a consumer of the one or more revised predicted identifiers.

16. The computer-implemented method of claim 12, wherein the first machine-learned model and the second machine-learned model are trained based at least in part on reinforcement learning.

17. A computer-implemented method for model training comprising:
receiving, by a computing system comprising one or more computing devices, training data, wherein the training data comprises one or more training features and one or more training identifiers, the one or more training features being associated with training medical data and the one or more training identifiers comprising one or more training medical codes;
training, by the computing system, a machine-learned model based at least in part on the one or more training features and the one or more training identifiers;
providing, by the computing system, input data to the machine-learned model, the input data comprising medical data;
receiving, by the computing system as an output of the machine-learned model, data indicative of one or more predicted identifiers, the one or more predicted identifiers comprising one or more predicted medical codes based at least in part on the medical data;
providing, by the computing system, the one or more predicted identifiers to a user device;
receiving, by the computing system via the user device, feedback data associated with the one or more predicted identifiers, wherein the feedback data comprises an indication that the one or more predicted medical codes are correct or incorrect; and
re-training, by the computing system, the machine-learned model based at least in part on the feedback data.

18. The computing system of claim 17, wherein training, by the computing system, the machine-learned model based at least in part on the one or more training features and the one or more training identifiers comprises:
providing, by the computing system, the one or more training features and the one or more training identifiers to the machine-learned model.

19. The computing system of claim 17, wherein the machine-learned model utilizes a semantic graph.

20. The computing system of claim 17, wherein re-training, by the computing system, the machine-learned model based at least in part on the feedback data comprises:
extracting, by the computing system, at least one of one or more feedback features or one or more feedback identifiers from the feedback data; and
providing, by the computing system, the one or more feedback features and the one or more feedback identifiers to the machine-learned model.
